(19) United States
(12) Patent Application Publication
(10) Pub. No.: US 2014/0026234 A1
(43) Pub. Date: Jan. 23, 2014

(54) BIOMARKERS AND THEIR USES IN CANCER DETECTION AND THERAPY

(71) Applicants: Michael P. Lisanti, Withington (GB); Sotgia Federica, Withington (GB)

(72) Inventors: Michael P. Lisanti, Withington (GB); Sotgia Federica, Withington (GB)

(73) Assignee: Thomas Jefferson University, Philadelphia, PA (US)

(21) Appl. No.: 13/982,038

(22) PCT Filed: Jan. 27, 2012
(86) PCT No.: PCT/US12/22933

§ 371 Date: Oct. 1, 2013

Related U.S. Application Data
(60) Provisional application No. 61/437,108, filed on Jan. 28, 2011.
(60) Provisional application No. 61/437,120, filed on Jan. 28, 2011.

(51) Int. Cl. G01N 33/574; A01K 67/027; G01N 33/50

(52) U.S. Cl. CPC G01N 33/57492; G01N 33/5023; A01K 67/0271
USPC 800/10; 506/12; 435/7.23; 424/133.1

(57) ABSTRACT

Presented herein are novel protein biomarkers related to cancers with stromal components. These newly identified biomarkers create the basis for multiple (single) assays using traditional bioassay technologies and when used in combination yield exceptional clinical sensitivity and specificity in the determination of diagnosis and/or prognosis of cancer. A new genetic model able to identify potential genetic suppressors and/or potential therapeutic agents for treating stromal cancers is also described. Means and methods for evaluating data generated using multiple biomarkers in order to validate findings and further the use of the biomarkers and the genetic model system in clinical, diagnostic and therapeutic uses is also included.

### FIELD OF THE INVENTION

The present invention relates to the biomarkers that are useful in the course of detection and/or treatment of cancer.

### BACKGROUND OF THE INVENTION

Cancer is one of the most significant diseases confronting mankind, and even though progress has been made in cancer treatment, particularly in the medical therapy of cancer, many challenges remain. In medical therapy of cancer, for example, the various anticancer agents for suppressing the growth of cancer cells that have been developed suppress the growth of not only cancer cells, but also normal cells, causing various side effects including nausea and vomiting, hair loss, myelosuppression, kidney damage, and nerve damage. Consequently, understanding the origins of these malignancies as well as developing models for the identification of new diagnostic arid therapeutic modalities is of significant interest to health care professionals.

Some research suggests that the environment around a cancer, for example, interstitial tissue which includes blood vessels, extracellular matrix (ECM), and fibroblasts, may play a role in the onset and progression of cancer. For example, Camps et al. (Proc. Natl. Acad. Sci. USA 1990, 87(1), 75-79) reported that when an athymic nude mouse was inoculated with tumor cells that do not form a tumor on their own or for which the tumor formation rate is low, together with tumorigenic fibroblasts, rapid and marked formation of a tumor was observed, and Olumi et al. (Cancer Res. 1999, 59(19), 5002-5011) reported that when peritumoral fibroblasts (i.e., cancer-associated fibroblasts or CAFs) from a prostate tumor patient were grafted on an athymic animal together with human prostate cells, neoplastic growth thereof was markedly accelerated. Furthermore, it has been clarified that a bioactive substance such as PDGF (platelet-derived growth factor), TGF-β (transforming growth factor-β), HGF (hepatocyte growth factor), or SDF-1 (stromal cell-derived factor-1) produced in the interstitium is involved in such growth of a tumor (Micke et al., Expert Opin Ther Targets. 2005, 9(6), 1217-1233).

Despite these findings, many needs remain unmet, including a better understanding of the environment around a cancer and effective models for the evaluation, diagnosis and generation of therapies for cancers, including metastatic cancers, in particular breast cancer. In this context, desirable models include those which provide insight into the processes underlying, for example, cancer onset and/or cancer progression, thereby facilitating diagnosis of cancer and/or generation of prophylactic and/or therapeutic treatments for cancer.

### BRIEF SUMMARY OF THE INVENTION

The present inventors have discovered a genetically tractable model system for identifying the genetic factors that govern the tumor promoting effects of cancer-associated fibroblasts. Accordingly, one aspect of the present invention provides a genetic model system for identifying the genetic factors that govern the tumor promoting effects of cancer-associated fibroblasts, the genetic model system comprising human Cav-1 deficient immortalized fibroblasts created using a targeted sh-RNA knock-down approach.

In certain embodiments, proteomics can be used to discover suitable biomarkers for use with the present invention. Proteomics is the study of proteome, the protein complement of the genome. The term proteome also used to refer to the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. by mass spectrometry and/or N-terminal sequencing, and (3) analysis of the data using bioinformatics. Proteomics methods are valuable supplements to other methods of gene expression profiling, and can be used, alone or in combination with other methods of the present invention, to detect the biomarkers of the present invention.

Accordingly, in another aspect, the present application concerns the identification, e.g., through proteomics, of one or more of a set of biomarkers (also referred to herein as “markers”) in tumor stroma that are predictive of the outcome of cancer in a cancer patient. These markers include ACO2, ALB, ANPEP, ANXA2, APEX1, ATP5A1, BAG2, CALR, CALU, CAPZB, CDC42, COL1A1, COL6A1, COL6A2, CRABP2, CRTAP, DMGDH, DNAJA3, DNM1L, ENO1, ETFB, FBN1, FKBP9, GAPDH, GDF2, GLUD1, HIST2H4B, HNRNPA2B1, HSPA8, HSPA9, HSPB1, HSPD1, IDH2, KIAA1409, LDHA, LDHAL6B, LGALS1, LGALS3, LMNA, MATR3, MT1M, MYL6, NDUFA5, NDUFS3, P4HA1, P4HA2, PITRM1, PKM2, PLOD1, PRDX1, PRDX4, PRDX6, PSME1, RAP1A, RCN1, RPLP2, S100A13, SCO2, SERPINH1, SHMT2, SOD2, SYNJ2BP, TPM1, TPM4, TRPC4AP, TXNDC5, UQCRFS1, VAT1, VIM, WDR78, XRCC6BP1, YWHAB and YWHAZ.

In another aspect, the present invention provides a method for determining the prognosis of a cancer in a subject, the method comprising: (a) determining the expression level of at least one biomarker or a prognostic signature, said at least one biomarker or prognostic signature being associated with the prognosis of the cancer, wherein said at least one biomarker or prognostic signature comprises one or more biological molecules associated with the prognosis of the cancer, in a cancer sample obtained from the subject; (b) comparing the expression level of the at least one biomarker or the prognostic signature in the cancer sample with the expression level of the at least one biomarker or the prognostic signature expression in a control sample, wherein said prognosis is made when the expression level of the at least one biomarker or a prognostic signature in the sample of cancer is greater than the expression level of the at least one biomarker or the prognostic signature in the control sample.

It has been discovered that loss of stromal Cav-1 in human cancer associated fibroblasts dramatically promotes the growth of stromal cancers. In particular, loss of stromal Cav-1 in human cancer associated fibroblasts dramatically promotes the growth of triple negative breast cancer cells (MDA-MB-231), increasing both tumor mass and tumor volume by about 4-fold, without any increase in angiogenesis.

It has also been discovered that the phenotype of the Cav-1 knock-down fibroblasts can be significantly reverted by reducing oxidative stress in the tumor micro-environment. In particular, it was found that mitochondrial superoxide disumutase 2 (SOD2) significantly reverted the tumor promoting phenotype of Cav-1 deficient fibroblasts. Loss of Cav-1 is believed to increases reactive oxygen species (ROS) production in stromal fibroblasts. To combat the resulting oxidative stress, SOD2 was stably overexpressed in Cav-1 knock-down fibroblasts using a lenti-viral vector with puromycin resistance. Also, as a control, Cav-1 knock-down cells were transfected with the empty vector alone, in parallel. Then, these two fibroblast lines were co-injected with MDA-MB-231 cells into the flanks of nude mice. Relative to the control, Cav-1 knock-down fibroblasts with over expressed SOD2 reduced the tumor promoting effects of Cav-1 knock-down fibroblasts by nearly 2-fold. Therefore, SOD2 is useful as a genetic suppressor of Cav-1 deficient stromal cancers.

Accordingly, in another aspect, the present invention provides a method for identifying genetic suppressors and/or genes or screening for potential therapeutic agents that reduce oxidative stress associated stromal Cav-1 deficient cancers. The method comprising: (a) providing a wild-type mouse injected into its flanks with a cancer cell line, wherein the cancer has a stromal component, as a control mouse; (b) providing a Cav-1 deficient mouse injected with a cancer cell line in its flanks, as a test mouse; (c) providing a potential therapeutic agent or a potential genetic suppressor; (d) injecting a placebo into a test mouse; (e) injecting a placebo into a control mouse; (f) treating both a test mouse and a control mouse with the potential therapeutic agent or the potential genetic suppressor; (g) measuring the mass and/or the size of the resulting cancer tumor in the test mouse and the control mouse in the presence of placebo; (h) measuring the mass and/or the size of the resulting cancer tumor in the test mouse and the control mouse in the presence of the potential therapeutic agent or the genetic suppressor; and (i) comparing the mass and/or the size in the test mouse with the mass and/or the size in the control mouse, in the presence of either placebo or the potential therapeutic agent or the potential genetic suppressor, wherein a decrease in the mass and/or the size in the test mouse injected with the potential therapeutic agent or the potential genetic identifies a therapeutic agent or a genetic suppressor which treats stromal Cav-1 deficient cancer.

It has been discovered that overexpression of one or more biological molecules is associated with aggressive disease and poor prognosis in cancer. Accordingly, in an exemplary embodiment, the present invention provides a biomarker (or a prognostic signature) for determining the risk of recurrence or progression of a cancer, the biomarker or the prognostic signature comprising a biological molecule or a combination of biomarkers associated with prognosis of the cancer and is selected from the group consisting of ACO2, ALB, ANPEP, ANXA2, APEX1, ATP5A1, BAG2, CALR, CALU, CAPZB, CDC42, COL1A1, COL6A1, COL6A2, CRABP2, CRTAP, DMGDH, DNAJA3, DNM1L, ENO1, ETFB, FBN1, FKBP9, GAPDH, GDF2, GLUD1, HIST2H4B, HNRNPA2B1, HSPA8, HSPA9, HSPB1, HSPD1, IDH2, KIAA1409, LDHA, LDHAL6B, LGALS1, LGALS3, LMNA, MATR3, MT1M, MYL6, NDUFA5, NDUFS3, P4HA1, P4HA2, PITRM1, PKM2, PLOD1, PRDX1, PRDX4, PRDX6, PSME1, RAP1A, RCN1, RPLP2, S100A13, SCO2, SERPINH1, SHMT2, SOD2, SYNJ2BP, TPM1, TPM4, TRPC4AP, TXNDC5, UQCRFS1, VAT1, VIM, WDR78, XRCC6BP1, YWHAB, YWHAZ.

### BRIEF DESCRIPTION OF THE DRAWINGS

FIG. 1 illustrates targeted knock-down of Cav-1 protein expression in hTERT-Fibroblasts;

FIG. 2 illustrates targeted knock-down of Cav-1 in stromal fibroblasts dramatically promotes breast cancer tumor growth;

FIG. 3 illustrates targeted knock-down of Cav-1 in stromal fibroblasts does not affect tumor angiogenesis;

FIG. 4 illustrates recombinant overexpression of eNOS in fibroblasts does not promote tumor growth;

FIG. 5 illustrates mitochondrial SOD2 significantly reverts the tumor promoting phenotype of Cav-1 deficient fibroblasts; and

FIG. 6 illustrates that cytoplasmic soluble SOD1 does not revert the tumor promoting phenotype of Cav-1 deficient fibroblasts.

### DETAILED DESCRIPTION OF THE INVENTION

A new xenograft system for modeling the lethality of a loss of stromal Cav-1 has been discovered. More specifically, it has been observed that a loss of stromal Cav-1 in human cancer associated fibroblasts dramatically promotes the growth of triple negative breast cancer cells (MDA-MB-231), increasing both tumor mass and tumor volume by about 4-fold, without any increase in angiogenesis. Furthermore, it has been shown that this phenotype can significantly reverted by reducing oxidative stress in the tumor micro-environment. The reduction of the oxidative stress was achieved via the recombinant overexpression of mitochondrially-targeted super-oxide dismutase (SOD2), in Cav-1 deficient cancer associated fibroblasts. As such, this new xenograft model provides a genetically tractable system for dissecting the key factors that govern the lethality of a Cav-1 deficient “pro-oxidative” tumor micro-environment.

According to a new paradigm for understanding tumor metabolism, called “The Autophagic Tumor Stroma Model of Cancer Metabolism” catabolism (autophagy) in the tumor stroma fuels the anabolic growth of aggressive cancer cells. It is believed that the tumor cells induce autophagy in adjacent cancer-associated fibroblasts via the loss of caveolin-1 (Cav-1), which is sufficient to promote oxidative stress in stromal fibroblasts. A human Cav-1 deficient immortalized fibroblasts created using a targeted sh-RNA knock-down approach have been used to demonstrate the role of Cav-1 deficient fibroblasts in promoting tumor growth. Relative to control fibroblasts, Cav-1 deficient fibroblasts dramatically promoted tumor growth in xenograft assays employing an aggressive human breast cancer cell line, namely MDA-MB-231 cells. Co-injection of Cav-1 deficient fibroblasts, with MDA-MB-231 cells, increased both tumor mass and tumor volume by about 4-fold.

Immuno-staining with CD31 indicated that this paracrine tumor promoting effect was independent of angiogenesis. Mechanistically, proteomic analysis of these human Cav-1 deficient fibroblasts identified >40 protein biomarkers that were upregulated, most of which were associated with (i) myofibroblast differentiation or (ii) oxidative stress/hypoxia.

In direct support of these findings, the tumor promoting effects of Cav-1 deficient fibroblasts could be functionally suppressed (nearly 2-fold) by the recombinant overexpression of SOD2 (superoxide dismutase 2), a known mitochondrial enzyme that de-activates superoxide, thereby reducing mitochondrial oxidative stress.

In contrast, cytoplasmic soluble SOD1 had no effect, further highlighting a specific role for mitochondrial oxidative stress in tumor promoting effect of Cav-1 deficient fibroblasts. The evidence directly support a key role for a loss of stromal Cav-1 expression and oxidative stress in cancer-associated fibroblasts, in promoting tumor growth, which is consistent with “The Autophagic Tumor Stroma Model of Cancer”. The human Cav-1 deficient fibroblasts described herein are a new genetically tractable model system for identifying suppressors of the cancer-associated fibroblast phenotype, via a genetic “complementation” approach.

The results disclosed herein elucidate the pathogenesis of triple negative and basal breasts cancers, as well as tamoxifen-resistance in ER-positive breast cancers, which are all associated with a Cav-1 deficient “lethal” tumor microenvironment, driving poor clinical outcome.

Accordingly, in some aspects, the present invention provides biomarkers and medical applications of the same, including methods of using the markers in diagnosis of cancer, determining prognosis of cancer, identifying potential cancer therapeutic agents, monitoring the progression of cancer in patients, and identifying genetic suppressors of cancer.

### I. BIOMARKERS

In one aspect the present invention provides a genetically tractable model system for identifying the genetic factors that govern the tumor promoting effects of cancer-associated fibroblasts. The genetic model system includes human fibroblast engineered to lack Cav-1, which in turn is co-injected with a human cancer cell line into immunodeficient mice.

In another aspect, the present invention provides a set of biomarkers or a prognostic signature associated with the prognosis of cancer, wherein in the set of biomarkers or the prognostic signature is selected from the group consisting of ACO2, ALB, ANPEP, ANXA2, APEX1, ATP5A1, BAG2, CALR, CALU, CAPZB, CDC42, COL1A1, COL6A1, COL6A2, CRABP2, CRTAP, DMGDH, DNAJA3, DNM1L, ENO1, ETFB, FBN1, FKBP9, GAPDH, GDF2, GLUD1, HIST2H4B, HNRNPA2B1, HSPA8, HSPA9, HSPB1, HSPD1, IDH2, KIAA1409, LDHA, LDHAL6B, LGALS1, LGALS3, LMNA, MATR3, MT1M, MYL6, NDUFA5, NDUFS3, P4HA1, P4HA2, PITRM1, PKM2, PLOD1, PRDX1, PRDX4, PRDX6, PSME1, RAP1A, RCN1, RPLP2, S100A13, SCO2, SERPINH1, SHMT2, SOD2, SYNJ2BP, TPM1, TPM4, TRPC4AP, TXNDC5, UQCRFS1, VAT1, VIM, WDR78, XRCC6BP1, YWHAB, YWHAZ and combinations thereof.

It has been discovered that levels of these biomarkers are significantly altered in human Cav-1 deficient fibroblasts, where Cav-1 deficient fibroblasts serve as a model for the “lethal tumor stroma” of human breast cancers having a poor clinical outcome. An increase in the expression level of any one or a panel of these biomarkers detected in a test biological sample compared to a normal control level indicates that the subject (from which/whom the sample was obtained) suffers from or is at risk of developing cancer.

### II. METHOD OF USE OF THE BIOMARKERS

In one embodiment, a deviation, increase or decrease in the expression level of any one or a panel of the above biomarkers detected in a test biological sample compared to a normal control level indicates that the subject (from which the sample was obtained) suffers from or is at risk of recurrence or progression cancer, such as breast cancer.

Alternatively, the expression level of any one or a panel of cancer-associated biomarkers in a biological sample may be compared to a cancer control level of the same biomarker or the same panel of biomarkers.

Thus, one aspect of the present invention is to provide a prognostic method for and/or treatment of cancer. In certain embodiments particular cancers having a stromal component/cells can be diagnosed and/or treated.

Stromal cells are connective tissue cells of an organ found in the loose connective tissue, including uterine mucosa (endometrium), prostate, bone marrow, bone marrow precursor cells, and the ovary and the hematopoietic system. The most common types of stromal cells include fibroblasts, immune cells, pericytes, endothelial cells, and inflammatory cells. Thus, cancers having a stromal component can occur in any organ or tissue with stromal component, including uterine mucosa (endometrium), prostate, bone marrow, bone marrow, the ovary and the hematopoietic system.

Accordingly, while any cancer can be considered within the scope of the present invention, particular cancers having a stromal component include leukemia, prostate cancer, ovarian sex cord-stromal cell cancers (e.g., Sertoli-Leydig cell tumor, granulosa-theca cell tumor, theca cell tumor, thecoma, fibroma, and gonadoblastoma), gastrointestinal stromal cancers (GIST), endometrial cancers, mesenchymal stromal cancers. In one embodiment, the cancer is breast cancer.

In one embodiment for diagnosing the presence or absence of a cancer, and in particular a cancer having a stromal component, includes the steps of: (a) providing a biological test sample from a subject afflicted with a cancer (e.g., breast cancer) or suspected of having cancer; (b) determining a level of at least one biomarker in the test sample that is associated with the prognosis of the cancer; (c) comparing the level of said at least one biomarker in the test sample to the level of the biomarker in a control sample, wherein an altered level, e.g., an increase or decrease in level, of the biomarker in said test sample relative to the level of the biomarker in said control sample is a prognostic indicator of the course of cancer disease in said subject.

In some embodiments the biomarker expression is increased or decreased 10%, 25%, 50% or more compared to the control level. Alternatively, biomarker expression is increased or decreased 1, 2, 3, 4, 5, 6, 7, or more, fold compared to the control level.

In some embodiments, the subject-derived biological sample may be any sample derived from a subject, e.g., a patient known to or suspected of having cancer. For example, the biological sample may be tissue containing sputum, blood, serum, plasma or cells from a breast tissue.

Another aspect of the present invention provides a method of monitoring the progression of breast cancer in a subject, the method comprising: (a) obtaining a first sample from a subject at a first time point and a second sample from said subject at a second time point; (b) determining the level of at least one biomarker in said first and second samples; (c) comparing the level of said at least one biomarker in said first sample to the level of said biomarker in said second sample, wherein an altered, e.g., elevated, level of the at least one biomarker in said second sample relative to the level in said first sample is an indication that the cancer has progressed in said subject.

One aspect of the present invention includes use of one or more isolated protein markers of the transformed fibroblasts, selected from a group consisting of ACO2, ALB, ANPEP, ANXA2, APEX1, ATP5A1, BAG2, CALR, CALU, CAPZB, CDC42, COL1A1, COL6A1, COL6A2, CRABP2, CRTAP, DMGDH, DNAJA3, DNM1L, ENO1, ETFB, FBN1, FKBP9, GAPDH, GDF2, GLUD1, HIST2H4B, HNRNPA2B1, HSPA8, HSPA9, HSPB1, HSPD1, IDH2, KIAA1409, LDHA, LDHAL6B, LGALS1, LGALS3, LMNA, MATR3, MT1M, MYL6, NDUFA5, NDUFS3, P4HA1, P4HA2, PITRM1, PKM2, PLOD1, PRDX1, PRDX4, PRDX6, PSME1, RAP1A, RCN1, RPLP2, S100A13, SCO2, SERPINH1, SHMT2, SOD2, SYNJ2BP, TPM1, TPM4, TRPC4AP, TXNDC5, UQCRFS1, VAT1, VIM, WDR78, XRCC6BP1, YWHAB, and YWHAZ for screening, detection, prognosis or determining therapeutic targets for cancer of the breast.

In another embodiment, the invention includes a method of screening, detecting, prognosticating cancer of the breast, comprising: isolating a set, i.e., two or more, of co-expressed differentiator marker proteins from the transformed fibroblast cells, wherein the co-expressed differentiator marker proteins being marker proteins selected from group consisting of ACO2, ALB, ANPEP, ANXA2, APEX1, ATP5A1, BAG2, CALR, CALU, CAPZB, CDC42, COL1A1, COL6A1, COL6A2, CRABP2, CRTAP, DMGDH, DNAJA3, DNM1L, ENO1, ETFB, FBN1, FKBP9, GAPDH, GDF2, GLUD1, HIST2H4B, HNRNPA2B1, HSPA8, HSPA9, HSPB1, HSPD1, IDH2, KIAA1409, LDHA, LDHAL6B, LGALS1, LGALS3, LMNA, MATR3, MT1M, MYL6, NDUFA5, NDUFS3, P4HA1, P4HA2, PITRM1, PKM2, PLOD1, PRDX1, PRDX4, PRDX6, PSME1, RAP1A, RCN1, RPLP2, S100A13, SCO2, SERPINH1, SHMT2, SOD2, SYNJ2BP, TPM1, TPM4, TRPC4AP, TXNDC5, UQCRFS1, VAT1, VIM, WDR78, XRCC6BP1, YWHAB, and YWHAZ.

In another embodiment, a method for treating a neoplastic disease in a patient is provided, comprising (a) obtaining a sample of stromal cells adjacent to the neoplasm from the neoplastic disease patient; (b) determining the level of caveolin-1 (Cav-1) protein expression in the stromal cells of the sample and comparing the level of Cav-1 protein expression in the stromal cells of the sample with the level of Cav-1 protein expression in a control; (c) predicting if the neoplasm will respond effectively to treatment with an anti-angiogenic agent, wherein said prediction is made when the level of Cav-1 protein expression in the stromal cells of the sample is lower than the level of Cav-1 protein expression in the control; and administering to said patient a therapeutically effective amount of an anti-angiogenic agent such as Avastin (bevacizumab).

In other embodiments, suitable anti-angiogenic agents include any agents that target the vascular endothelial growth factor (VEGF), platelet-derived growth factor (PDGF), and fibroblast growth factor (FGF) pathways in patients with cancer. Examples the anti-angiogenic agents include selective inhibitors of the VEGF pathway (e.g., bevacizumab and VEGF Trap); VEGF/PDGF pathway inhibitors (e.g., sorafenib and sunitinib); and VEGF/PDGF/FGF pathway inhibitors (e.g., cediranib, pazopanib, and BIBF 1120)

In another embodiment, the invention provides a method of predicting whether a cancer in a cancer patient will respond effectively to treatment with an anti-angiogenic agent, comprising: (a) obtaining a sample of stromal cells adjacent to the cancer in a sample derived from the cancer patient; (b) determining the level of Cav-1 protein expression in the stromal cells of the sample; and (c) comparing the level of Cav-1 protein expression in the stromal cells of the sample with the level of Cav-1 protein expression in a control; (c) predicting if the cancer will respond effectively to treatment with an anti-angiogenic agent, wherein low expression levels of Cav-1 protein expression in the stromal layers relative to Cav-1 expression levels in the control correlate with a cancer that will not respond effectively to treatment with an angiogenic agent.

In another aspect, the present invention provides a method for identifying genetic suppressors that can block the tumor promoting properties of cancer-associated fibroblast cells specifically lacking Cav-1. The method comprises identifying genes which when expressed or undergo modulated expression reduce oxidative stress.

In another embodiment, the present invention includes a method of modulating one or more biomarker which are indicative of a disease state to thereby treat of cancer. In such an embodiment, one or more biomarkers can be modulated by the administration a compound which reduces oxidative stress. In one embodiment, acetylcysteine (commonly referred to as N-acetylcysteine) can be used.

It should be noted that the predictive value of stromal Cav-1 is independent of epithelial marker status, and is effective in all the major sub-types of breast cancer, including ER+, PR+, HER2+and triple negative (ER−, PR−, HER2−) breast cancer patients. In triple negative (TN) patients, which is one of the most lethal types of breast cancer, stromal Cav-1 effectively distinguished between low-risk and high-risk patients.

In an embodiment, the predictive value of stromal Cav-1 is independent of epithelial marker status, and is effective any cancer selected from the group consisting of basal cell carcinoma, glioma, breast cancer, chondrosarcoma, colon cancer, esophageal cancer, gastric cancer, gastrointestinal stromal tumor, hepatocellular cancer, lung cancer, medulloblastoma, melanoma, neuroectodermal tumors, osteogenic sarcoma, ovarian cancer, pancreatic cancer, prostate cancer, and testicular cancer.

In TN patients with high stromal Cav-1, their overall survival was >75% up to 12 years post-diagnosis. In contrast, in TN patients with absent stromal Cav-1, their overall survival was <10% at 5 years post-diagnosis. Thus, a loss of stromal Cav-1 is a key predictor of a “lethal” tumor micro-environment.

Accordingly, in an aspect, the present invention provides a method of identifying a potential therapeutic agent that treats stromal Cav-1 deficient cancer comprising: (a) providing a wild-type animal, e.g., a mouse, injected with mouse mammary cancer cells in the mammary fat pad as a control mouse; (b) providing a Cav-1 deficient mouse injected with mouse mammary cancer cells in the mammary fat pad as a test mouse; (c) providing a potential therapeutic agent; (d) injecting a placebo into a test mouse; (e) injecting a placebo into a control mouse; (f) treating both a test mouse and a control mouse with the potential therapeutic agent; (g) measuring the mass and/or the size of the resulting cancer tumor in the test mouse and the control mouse in the presence of placebo; (h) measuring the mass and/or the size of the resulting cancer tumor in the test mouse and the control mouse in the presence of the potential therapeutic agent; and (i) comparing the mass and/or the size in the test subject mouse with the mass and/or the size in the control mouse, in the presence of either placebo or the potential therapeutic agent, wherein a decrease in the mass and/or the size in the test mouse injected with the potential therapeutic agent identifies a therapeutic agent which treats stromal Cav-1 deficient cancer.

A “normal control level” indicates an expression level of a biomarker detected in a normal, healthy individual or in a population of individuals known not to be suffering from breast cancer. A control level is a single expression pattern derived from a single reference population or from a plurality of expression patterns. In contrast to a “normal control level”, the “control level” is an expression level of a biomarker detected in an individual or a population of individuals whose background of the disease state is known (i.e., cancerous or non-cancerous). Thus, the control level may be determined based on the expression level of a biomarker in a normal, healthy individual, in a population of individuals known not to be suffering from breast cancer, a patient suffering from breast cancer or a population of the patients. The control level corresponding to the expression level of a biomarker in a patient of breast cancer or a population of the patients are referred to as “breast cancer control level”. Furthermore, the control level can be a database of expression patterns from previously tested individuals.

### III. EXAMPLES

The Examples that follow are illustrative of specific embodiments of the invention, and various uses thereof. They set forth for explanatory purposes only, and are not to be taken as limiting the invention.

General Techniques

Cell Cultures

Human immortalized fibroblasts (hTERT-BJ1) and human breast cancer cells (MDA-MB-231-GFP) were grown in Dulbecco's modified Eagle's medium (DMEM) containing 10% fetal bovine serum in a 37° C., 5% CO2 incubator. hTERT-BJ1 fibroblasts stably-expressing eNOS, and the corresponding vector alone control, were prepared as previously described in Martinez-Outschoorn et al. Cell Cycle 2010, 9, 3256-3276.

sh-RNA silencing and retroviral infection. sh-RNA control and two pre-designed sh-RNAs targeting nucleotides 383-403 (5′-GCT GAG CGA GAA GCA AGT GTA-3′) or 660-680 (5′-TGG GCA GTT GTA CCA TGC ATT-3′) of the CAV1 mRNA (NM_{—}001753.3) were obtained from Invitrogen and were subcloned into the pQCXIP-GFP retroviral vector (Clontech, Inc.). The sh-RNA negative control contains an insert that forms a hairpin structure that is predicted not to target any known vertebrate gene (Invitrogen, Inc.). For retroviral infection, vectors were transiently transfected into the amphotropic Phoenix packaging cell line, using a modified calcium phosphate method. Forty-eight hours post-transfection, the viral supernatant was collected, 0.45 μm sterile filtered, and added to the target cells. Two infection cycles were carried out (every 12 hours) with hTERT-BJ1 cells. Effective knockdown of CAV1 was determined by Western blot analysis of FACS-sorted GFP-positive cells.

sh-RNA Silencing and Retroviral Infection

sh-RNA control and two pre-designed sh-RNAs targeting nucleotides 383-403 (5′-GCT GAG CGA GAA GCA AGT GTA-3′) or 660-680 (5′-TGG GCA GTT GTA CCA TGC ATT-3′) of the CAV1 mRNA (NM_{—}001753.3) were obtained from Invitrogen and were subcloned into the pQCXIP-GFP retroviral vector (Clontech, Inc.). The sh-RNA negative control contains an insert that forms a hairpin structure that is predicted not to target any known vertebrate gene (Invitrogen, Inc.). For retroviral infection, vectors were transiently transfected into the amphotropic Phoenix packaging cell line, using a modified calcium phosphate method. Forty-eight hours post-transfection, the viral supernatant was collected, 0.45 μm sterile filtered, and added to the target cells. Two infection cycles were carried out (every 12 hours) with hTERT-BJ1 cells. Effective knockdown of CAV1 was determined by Western blot analysis of FACS-sorted GFP-positive cells.

Recombinant Expression of SOD2

hTERT-BJ1 Cav-1 knock-down cells were transduced with a lenti-viral vector encoding SOD2 (Human superoxide dismutase 2, mitochondrial; Accession #'sY00985.1/NM_{—}000636.2), with puromycin resistance (pReceiver-I0569-Lv105) or with the vector alone (pReceiver-Lv105), as a critical negative control (GeneCopoeia, Inc.). The same strategy was also used to overexpress SOD1 (Human superoxide dismutase 1, soluble; Accession #X02317), with the construct pReceiver-K2710-Lv105 (GeneCopoeia, Inc.), Athymic Ncr-nu/nu mice (7-to-9 weeks old) were purchased from Taconic. For each injection, 106 MDA-MB-231 cells and 300,000 hTERT-BJ1 fibroblasts in 100 μl of sterile PBS were injected subcutaneously into the flanks of the nude mice. Two flank injections were performed per mouse. Tumors weights and volumes were then measured at 4,5 weeks post-injection (Chiavarina et al., Cell Cycle 2010, 9, 3534-3551; Bonuccelli et al. Cell Cycle 2010, 9, 1960-1971; Migneco et al. Cell Cycle 2010, 9, 2412-2422; and Bonuccelli et al., Cell Cycle 2010, 9, 3506-3514).

Quantitation of Tumor Angiogenesis

Immunohistochemical staining for CD31 was performed on frozen tumor sections using a 3-step biotin-streptavidin-horseradish peroxidase method. Frozen tissue sections (6 μm) were fixed in 2% paraformaldehyde in PBS for 10 min and washed with PBS. After blocking with 10% rabbit serum the sections were incubated overnight at 4° C. with rat antimouse CD31 antibody (BS Biosciences) at a dilution of 1:200, followed by biotinylated rabbit anti-rat IgG (1:200) antibody and streptavidin-HRP. Immunoreactivity was revealed with 3,3′-diaminobenzidine. For quantitation of vessels, CD31-positive vessels were enumerated in 4-6 fields within the central area of each tumor using a 20× objective lens and an ocular grid (0.25 mm2 per field). The total number of vessels per unit area was calculated and the data was represented graphically.

Protcomic Analysis

2-D DICE (two-dimensional difference gel electrophoresis) and mass spectrometry protein identification were run by Applied Biomics (Hayward, Calif.). Image scans were carried out immediately following the SDS-PAGE using Typhoon TRIO (Amersham BioSciences) following the protocols provided. The scanned images were then analyzed by Image QuantTL software (GE-Healthcare), and then subjected to in-gel analysis and cross-gel analysis using DeCyder software version 6.5 (GE-Healthcare). The ratio of protein differential expression was obtained from in-gel DeCyder software analysis. The selected spots were picked by an Ettan Spot Picker (GE-Healthcare) following the DeCyder software analysis and spot picking design. The selected protein spots were subjected to in-gel trypsin digestion, peptides extraction, desalting and followed by MALDI-TOF/TOF (Applied Biosystems) analysis to determine the protein identity.

Proteomic analysis of Cav-1 deficient fibroblasts provides evidence for the onset of a myofibroblast phenotype and mitochondrial oxidative stress. In order to mechanistically dissect the tumor promoting activity associated with human Cav-1 knockdown fibroblasts, they were subjected to an extensive unbiased proteomic analysis, as detailed above. Cav-1 deficient fibroblasts showed the upregulation of 15 gene products associated with the myo-fibroblast phenotype, including numerous proteins associated with collagen synthesis and processing (COL1A1; COL6A1/2; P4HA1/2; HSP47; PLOD1), muscle-specific proteins (VIM; MYL6; TPM1/4), and other components of the extracellular matrix (LGALS1/3; CRTAP) (see Table 1).

While not wishing to be bound by any particular mechanism of action or theory, the observed upregulation of factors associated with the autophagic destruction of mitochondria (DNM1L), and lipid/protein catabolism (DMGDH; PSME1) is in accordance with the idea that oxidative stress induces autophagy/mitophagy. The upregulation of CDC42 is consistent with the activation of ROS production via NADPH oxidase and the overexpression of DMGDH (dimethylglycine dehydrogenase) is also consistent with oxidative stress. DMGDH functions in the catabolism of choline, catalyzing the oxidative demethylation of dimethylglycine to form sarcosine. Increase in sarcosine directly parallels a loss of reduced glutathione, indicative of an oxidative tumor micro-environment. Thus, increased sarcosine is a biomarker for oxidative stress, which is associated with advanced prostate cancer and the development of metastatic disease. Furthermore, observed upregulation of 4 calcium-binding proteins (CALU; CALR; RCN1; S100A13), suggests that there may be dys-regulation of calcium homeostasis, in Cav-1 deficient fibroblasts (see Table 1).

Since oxidative stress is known to be associated with the disruption of calcium homeostasis, the upregulation of both myofibroblast and oxidative stress markers accords with oxidative stress as being sufficient and/or required for the induction of the myofibroblast phenotype. Also, many of the proteins induced by Cav-1 knock-down in fibroblasts are highly expressed in the tumor stroma of human breast cancer patients, and are associated with tumor recurrence or metastasis (See Table 2).

TABLE 1
Proteomic analysis of hTERT Cav-1 knock-down (KD) fibroblasts

| | Fold change (Cav-1 | | Protein spot |
|---|---|---|---|
| | KD/Control) | Accession number | number |
| Myo-fibroblast Associated Proteins and Extracellular Matrix | | | |
| collagen, type VI, alpha1 (COL6A1) | 3.28 | gi\|87196339 | 3 |
| collagen, type VI, alpha1 (COL6A1) | 3.10 | gi\|87196339 | 2 |
| collagen, type VI, alpha2 (COL6A2) | 2.38 | gi\|115527062 | 7 |
| collagen, type VI, alpha2 (COL6A2) | 1.68 | gi\|115527062 | 6 |
| lectin, galactoside-binding, soluble, 3 (Galectin-3; LGAL53) | 2.04 | gi\|134104936 | 49 |
| lectin, galactoside-binding, soluble, 3 (Galectin-3; LGAL53) | 2.10 | gi\|157829667 | 50 |
| lectin, galactoside-binding, soluble, 1 (Galectin-1; LGAL51) | 1.49 | gi\|42542977 | 63 |
| prolyl 4-hydroxylase, alpha polypeptide I (P4HA1) | 1.83 | gi\|63252888 | 23 |
| prolyl 4-hydroxylase, alpha polypeptide II (P4HA2) | 1.34 | gi\|119582749 | 22 |
| serpin peptidase inhibitor, clade H (heat shock protein 47), member 1, (collagen | 1.93 | gi\|32454741 | 38 |
| binding protein 1) (SERPINH1) (rheumatoid arthritis-related antigen RA-A47; | | | |
| HSP47) | | | |
| vimentin (VIM) | 1.76 | gi\|62414289 | 31 |
| vimentin (VIM) | 1.67 | gi\|340219 | 30 |
| vimentin (VIM) | 1.50 | gi\|340219 | 33 |
| vimentin (VIM) | 1.35 | gi\|340219 | 32 |
| tropomyosin 4 (TPM4) | 1.69 | gi\|4507651 | 24 |
| tropomyosin 4 (TPM4) | 1.58 | gi\|4507651 | 45 |
| tropomyosin 4 (TPM4) | 1.54 | gi\|4507651 | 44 |
| tropomyosin 1 (alpha) (TPM1) | 1.31 | gi\|339956 | 43 |
| collagen; type 1, alpha1 (COL1A1) | 1.65 | gi\|110349772 | 4 |
| collagen, type 1, alpha1 (COL1A1) | 1.62 | gi\|186893270 | 5 |
| myosin, light chain 6, alkali, smooth muscle and non-muscle (MYL6) | 1.44 | gi\|17986258 | 60 |
| myosin, light chain 6, alkali, smooth muscle and non-muscle (MYL6) | 1.42 | gi\|119617307 | 59 |
| procollagen-lysine 1,2-oxoglutarate 5-dioxygenase 1 (PLOD1) (lysyl hydroxylase) | 1.39 | gi\|190074 | 15 |
| fibrillin (FBN1) | 1.38 | gi\|306746 | 39 |
| cartilage associated protein (CRTAP) | 1.34 | gi\|5453601 | 34 |
| Oxidative Stress/ROS Production, Hypoxia, Mitochondrial Metabolism and Glycolysis | | | |
| cellular retinoic acid binding protein 2 (CRABP2) (induced by hypoxia and/or | 4.40 | gi\|6730582 | 62 |
| oxidative stress) | | | |
| synaptojanin 2 binding protein (SYNJ2BP) mitochondrial outer membrane protein | 2.85 | gi\|152149141 | 61 |
| 25 (induced by hypoxia and/or oxidative stress) | | | |
| NADH dehydrogenase (ubiquinone) 1alpha subcomplex, 5, 13 kDa (NDUFA5) (mito | 2.75 | gi\|119603997 | 57 |
| complex I) (ROS Production) | | | |
| NADH dehydrogenase (ubiquinone) Fe—S protein 3, 30 kDa (NADH-coenzyme Q | 1.83 | gi\|5138999 | 46 |
| reductase) (NDUF53) (mito complex I) (ROS Production) | | | |
| ubiquinol-cytochrome c reductase (mito complex III), Rieske Iron-sulfur polypep- | 1.46 | gi\|54036562 | 48 |
| tide 1 (UQCRFS1) (ROS Production) | | | |
| electron-transfer-flavoprotein, beta polypeptide (ETFB) (ROS Production) | 2.10 | gi\|62420877 | 50 |
| heat shock 27 kDa protein 1 (HSPB1) (Induced by hypoxia and/or oxidative stress) | 1.83 | gi\|662841 | 46 |
| cell division cycle 42 (GTP binding protein, 25 kDa) (CDC42) (promotes oxidative | 1.65 | gi\|5542168 | 51 |
| stress/ROS Production) | | | |
| peroxiredoxin 4 (PRDX4), anti-oxidant | 1.51 | gi\|5453549 | 47 |
| peroxiredoxin 1 (PRDX1), anti-oxidant | 1.51 | gi\|55959888 | 47 |
| alanyl (membrane) aminopeptidase (ANPEP) (glutathione metabolism) | 1.50 | gi\|157266300 | 1 |
| glutamate dehydrogenase 1 (GLUD1) (induced by hypoxia and/or oxidative stress) | 1.50 | gi\|20151189 | 28 |
| glyceraldehyde-3-phosphate dehydrogenase (GAPDH) (aging-associated gene 9 | 1.45 | gi\|31645 | 40 |
| protein; glycolysis) (Induced by hypoxia and/or oxidative stress) | | | |
| lactate dehydrogenase A (LDHA; glycolysis) (induced by hypoxia and/or oxidative | 1.31 | gi\|13786849 | 41 |
| stress) | | | |
| mitochondrial heat shock 60 kD protein 1 (HSP60; HSPD1) (induced by oxidative | 1.42 | gi\|189502784 | 21 |
| stress; protects Fe—S proteins) | | | |
| isocitrate dehydrogenase 2 (NADP [symbol] ), mitochondrial (IDH2) (induced by oxidative | 1.38 | gi\|62897391 | 39 |
| stress) | | | |
| annexin A2 (ANXA2) (induced by hypoxia and/or oxidative stress) | 1.34 | gi\|18645167 | 41 |
| thioredoxin domain containing 5 (endoplasmic reticulum) (TXNDC5), anti-oxidant | 1.34 | gi\|30354488 | 34 |
| (hypoxia-induced; protects hypoxic cells against apoptosis) | | | |
| metallothionein 1M (MT1M) (induced by hypoxia and/or oxidative stress) | 1.32 | gi\|28866966 | 14 |
| DNA Damage and Repair | | | |
| Ku70-binding protein (KUB3; XRCC68P1) (DNA double-strand break repair) | 1.40 | gi\|4867999 | 64 |
| (induced by oxidative stress) | | | |
| Mitochondial Fission and the Autophagic Destruction of Mitochondria | | | |
| dynamin 1-like (DNM1L); mitochondrial fission (Reduces oxidative stress via | 2.61 | gi\|171460918 | 52 |
| Mitophagy) | | | |
| Lamin A/C: Mutations Promote Susceptibility to Oxidative Stress | | | |
| lamin A/C (LMNA); progerin | 1.66 | gi\|5031875 | 19 |
| lamin A/C (LMNA); progerin | 1.63 | gi\|5031875 | 18 |
| lamin A/C (LMNA); progerin | 1.58 | gi\|57014047 | 16 |
| lamin A/C (LMNA); progerin | 1.55 | gi\|5031875 | 20 |
| lamin A/C (LMNA); progerin | 1.52 | gi\|5031875 | 17 |
| Lipid and Protein Catabolism | | | |
| dimethylglycine dehydrogenase, mitochondrial (DMGDH) (catabolism of choline, | 1.38 | gi\|18490229 | 39 |
| catalyzing the oxidative demethylation of dimethylglycine to form sarcosine) | | | |
| (protective against hypoxia-induced-apoptosis) | | | |
| proteasome (prosome, macropain) activator subunit 1 (PA28alpha) (PSME1) | 1.35 | gi\|5453990 | 66 |
| Calcium Binding Proteins | | | |
| calumenin (CALU) | 1.72 | gi\|49456627 | 29 |
| calreticulin (CALR) | 1.62 | gi\|62897681 | 12 |
| reticulocalbin 1, EF-hand calcium binding domain (RCN1) (proliferation-inducing | 1.41 | gi\|4506455 | 35 |
| gene 20) | | | |
| S100 calcium binding protein A13 (S100A13) | 1.40 | gi\|82407535 | 64 |
| Protein Expression/Synthesis and Folding | | | |
| FK506 binding protein 9, 63 kDa (FKBP9) (peptidyl-prolyl cis-trans isomerase) (pro- | 1.95 | gi\|33469985 | 11 |
| tein folding) | | | |
| splicing factor, arginine/serine-rich 3 (SFRS3) (RNA processing) | 1.65 | gi\|4506901 | 51 |
| ribosomal protein, large, P2 (RPLP2) (protein synthesis) | 1.71 | gi\|4506671 | 58 |
| Signaling Proteins | | | |
| RAP1A, member of RAS oncogene family (RAP1A) | 1.83 | gi\|14595132 | 46 |
| tyrosine 3-monooxygenase/tryptophan 5-monooxygenase activation protein, | 1.45 | gi\|4507949 | 67 |
| beta polypeptide (14-3-3 protein beta/alpha; YWHAB) | | | |
| tyrosine 3-monooxygenase/tryptophan 5-monooxygenase activation protein, | 1.45 | gi\|4507953 | 67 |
| zeta polypeptide (14-3-3 protein zeta; YWHAZ) | | | |
| Other | | | |
| KIAA1409 (a hypoxia-responsive gene) | 4.10 | gi\|119601937 | 65 |

[symbol]

indicates data missing or illegible when filed

TABLE 2
Intersection of Cav-1 deficient fibroblast proteomics with
the transcriptome of human breast cancer tumor stroma

| | | Recurrence- | Metastasis- |
|---|---|---|---|
| Gene symbol | Tumor stroma | Prone stroma | Prone stroma |
| ANPEP | | | |
| ANXA2 | | | |
| CALR | | 3.60E−02 | |
| CALU | 9.97E−07 | | |
| CDC42 | 2.46E−22 | 1.36E−03 | |
| COL1A1 | 3.20E−17 | 2.46E−03 | |
| COL6A1 | 8.97E−19 | | 4.00E−02 |
| COL6A2 | | | |
| CRA8P2 | | | |
| CRTAP | 3.34E−12 | 4.64E−02 | 4.45E−02 |
| DMGDH | 4.61E−03 | | 2.88E−02 |
| DNM1L | | | |
| ETFB | | | |
| FBN1 | 1.22E−20 | | |
| FKBP9 | 4.73E−16 | | |
| GAPDH | | | |
| GLUD1 | | | |
| HSPB1 | | | 9.27E−04 |
| HSPD1 | | | |
| IDH2 | | | |
| KIAA1409 | | | |
| LDHA | | | |
| LDHAL6B | 3.67E−10 | | |
| LGAL51 | | | |
| LGAL53 | 3.92E−03 | | |
| LMNA | | | |
| MT1M | | | |
| MYL6 | 4.15E−10 | | |
| NDUFA5 | | | |
| NDUF53 | | | |
| P4HA1 | | | |
| P4HA2 | 1.72E−11 | 6.06E−03 | 2.63E−02 |
| PLOD1 | | | |
| PRDX1 | | | |
| PRDX4 | | | |
| PSME1 | | | |
| RAP1A | | | |
| RGN1 | | | |
| RPLP2 | | | |
| S100A13 | 1.32E−13 | | 1.90E−02 |
| SERPINH1 | | | |
| SYNJ2BP | | | |
| TPM1 | 2.20E−26 | 5.23E−07 | |
| TPM4 | 7.86E−15 | 1.68E−05 | |
| TXNDC5 | | | |
| UQCRF5 [symbol] | | | |
| VIM | | | |
| XRCC6BP1 | 4.52E−13 | 1.64E−02 | |
| YWHAB | | | 2.78E−02 |
| YWHAZ | 1.26E−20 | | |

Proteins that were transcriptionally upregulated in laser-capture microdissected human breast cancer tumor stroma are shown in BOLD. Those gene products that are associated with tumor recurrence or metastasis are shown in BOLD and are underlined. LDHA was not found to be transcriptionally upregulated; however, its close relative LDHAL6B was transcriptionally increased in tumor stroma. P values are as shown. [symbol]
indicates data missing or illegible when filed

Proteomic Analysis of hTERT eNOS Fibroblasts

Proteomic analysis of eNOS transfected fibroblasts identified SOD2 as a potential stromal tumor suppressor (FIG. 4 and Tables 3 and 4) due to its ability to combat mitochondrial oxidative stress. SOD2 (super oxide dismutase 2) is an enzyme that is specifically localized to mitochondria and detoxifies super-oxide, resulting in decreased oxidative stress. Overexpression of eNOS phenocopies many of the effects observed due to loss of Cav-1 because Cav-1 normally functions as an inhibitor of NOS, thereby preventing NO production. Proteomic analysis of eNOS-fibroblasts (hTERT-BJ1 fibroblasts stably-overexpressing eNOS) produced a similar proteomic profile (see Table 3) as observed with Cav-1 knock-down fibroblasts (see Table 1), with the simultaneous upregulation of both myofibroblast markers and proteins associated with oxidative stress/hypoxia.

The eNOS proteomic data is also consistent with analyses of several other Cav-1 deficient fibroblastic cell lines, including murine Cav-1 (−/−) knock-out fibroblasts, murine mesenchymal stem cells, HIF-alpha and IKBKE-transfected hTERT-BJ1 human fibroblasts, which also lack Cav-1. Thus, the induction of mitochondrial oxidative stress with eNOS is indeed sufficient to induce a proteomic profile similar to the one we observed due to a loss of Cav-1.

This observation supports the idea that a loss of Cav-1 leads to increased NO production. However, unlike Cav-1 deficient fibroblasts (FIG. 2), eNOS overexpressing fibroblasts did not promote tumor growth (FIG. 4). FIG. 2 illustrates targeted knock-down of Cav-1 in stromal fibroblasts dramatically promotes breast cancer tumor growth. Control or Cav-1 knock-down fibroblasts (300,000 cells) were co-injected with MDAMB-321 cells (1 million cells) in the flanks of nude mice. After 4.5 weeks post-injection, the tumors were harvested. Relative to control fibroblasts, Cav-1 knock-down fibroblasts increased tumor mass by about 4-fold (A) and increased tumor volume by about 4-fold (B). An asterisk indicates that p≦0.01. Fibroblasts injected alone did not form tumors. MDA-MB-231 cells injected alone, behaved as MDA-231 cells injected with control fibroblasts. N=20 flank injections for each experimental group (Ctl=control sh-RNA; sh-Cav1=harboring sh-RNA targeting Cav-1).

FIG. 4 illustrates recombinant overexpression of eNOS in fibroblasts does not promote tumor growth. Control or eNOS-overexpressing fibroblasts (300,000 cells) were co-injected with MDA-MB-321 cells (1 million cells) in the flanks of nude mice. After 4 weeks post-injection, the tumors were harvested. No significant differences were noted between control fibroblasts and eNOS-overexpressing fibroblasts. N=10 flank injections for each experimental group (n.s.=not significant; EV=empty vector; eNOS=stably overexpressing eNOS; (A)=tumor weight; (B)=tumor volume).

Thus, it would appear that eNOS overexpressing fibroblasts may have upregulated a “tumor suppressor protein” to allow them to adjust or adapt to a very high-level of mitochondrial oxidative stress, thereby “repressing” their tumor promoting activity. The sequence listings associated with the accession numbers set forth in Table 3 are hereby incorporated by reference as if set forth fully herein.

TABLE 3
Proteomic analysis of hTERT eNOS fibroblasts

| | Fold change (eNOS/ | | Protein spot |
|---|---|---|---|
| | Control) | Accession number | number |
| Myo-fibroblast Associated Proteins and Extracellular Matrix | | | |
| vimentin (VIM) | 2.20 | gi\|62414289 | 22 |
| vimentin (VIM) | 1.47 | gi\|62414289 | 23 |
| growth differentiation factor 2 (GDF2) | 1.85 | gi\|7705308 | 13 |
| albumin (ALB) | 1.70 | gi\|11493459 | 14 |
| capping protein (actin filament) muscle Z-line, beta (CAPZB) | 1.65 | gi\|54695812 | 44 |
| collagen, type VI, alpha2 (COL6A2) | 1.49 | gi\|115527062 | 3 |
| pitrilysin metallopeptidase 1 (PITRM1) | 1.43 | gi\|66267592 | 5 |
| Oxidative Stress/ROS Production, Hypoxia, Mitochondrial Metabolism and Glycolysis | | | |
| glyceraldehyde-3-phosphate dehydrogenase (GAPDH) | 1.75 | gi\|31645 | 42 |
| glyceraldehyde-3-phosphate dehydrogenase (GAPDH) | 1.45 | gi\|31645 | 43 |
| heterogeneous nuclear ribonucleoprotein A2/B1 (HNRNPA2B1) | 1.65 | gi\|14043072 | 44 |
| enolase 1, (alpha) (ENO1) | 1.57 | gi\|4503571 | 27 |
| histone cluster 2, H4b (HIST2H4B) | 1.53 | gi\|12450316 | 57 |
| peroxiredoxin 6 (PRDX6) | 1.52 | gi\|4758638 | 47 |
| SCO cytochrome oxidase deficient homolog 2 (yeast) (SCO2) (chaperone for | 1.52 | gi\|153791313 | 47 |
| mitochondrial cytochrome c oxidase subunit II (COX2)) (mito complex IV of the | | | |
| electron transport chain) | | | |
| annexin A2 (ANXA2) | 1.50 | gi\|56966699 | 40 |
| annexin A2 (ANXA2) | 1.48 | gi\|56966699 | 39 |
| annexin A2 (ANXA2) | 1.45 | gi\|4757756 | 38 |
| vesicle amine transport protein 1 homolog (T. californica) (VAT1) (member of the | 1.47 | gi\|18379349 | 26 |
| quinone oxidoreductase subfamily) | | | |
| superoxide dismutase 2, mitochondrial (SOD2) | 1.44 | gi\|30841309 | 48 |
| serine hydroxymethyltransferase 2 (mitochondrial) (SHMT2) | 1.40 | gi\|746436 | 29 |
| ATP synthase, H [symbol] transporting, mitochondrial F1 complex, alpha subunit 1, cardiac | 1.40 | gi\|4757810 | 29 |
| muscle (ATP5A1) | | | |
| pyruvate kinase, muscle (PKM2) | 1.41 | gi\|119598292 | 18 |
| pyruvate kinase, muscle (PKM2) | 1.38 | gi\|67464392 | 19 |
| glutamatedehydrogenase 1 (GLUD1) | 1.41 | gi\|20151189 | 28 |
| aconitase 2, mitochondrial (ACO2) | 1.36 | gi\|4501867 | 8 |
| DNA Damage and Repair | | | |
| APEX nuclease (multifunctional DNA repair enzyme) 1 (APRX1) (repairs oxidative | 1.45 | gi\|299037; | 43 |
| DNA damage) | | gi\|219478 | |
| Stress Associated Proteins | | | |
| heat shock 70 kDa protein 8 (HSPA8) | 1.99 | gi\|5729877 | 12 |
| heat shock 70 kDa protein 9 (mortalin) (HSPA9) (proliferation and cellular aging) | 1.53 | gi\|21040386 | 11 |
| BCL2-associated athanogene 2 (BAG2) | 1.37 | gi\|49065418 | 46 |
| DnaJ (Hsp40) homolog, subfamily A, member 3 (DNAJA3) | 1.37 | gi\|159164245 | 46 |
| TNFalpha/NFκB Signaling | | | |
| transient receptor potential cation channel, subfamily C, member 4 associated | 1.52 | gi\|12654951 | 47 |
| protein (TRPC4AP) (activation of the NFκB1 response) (scaffolding protein to link | | | |
| TNFRSF1A to the IKK signalosome) | | | |
| Other | | | |
| WD repeat domain 78 (WDR78) | 1.85 | gi\|55665586 | 13 |
| matrin 3 (MATR3) | 1.43 | gi\|21626466 | 5 |

Note that SOD2, a potential tumor suppressor, is highlighted in BOLD and is underlined. [symbol]
indicates data missing or illegible when filed

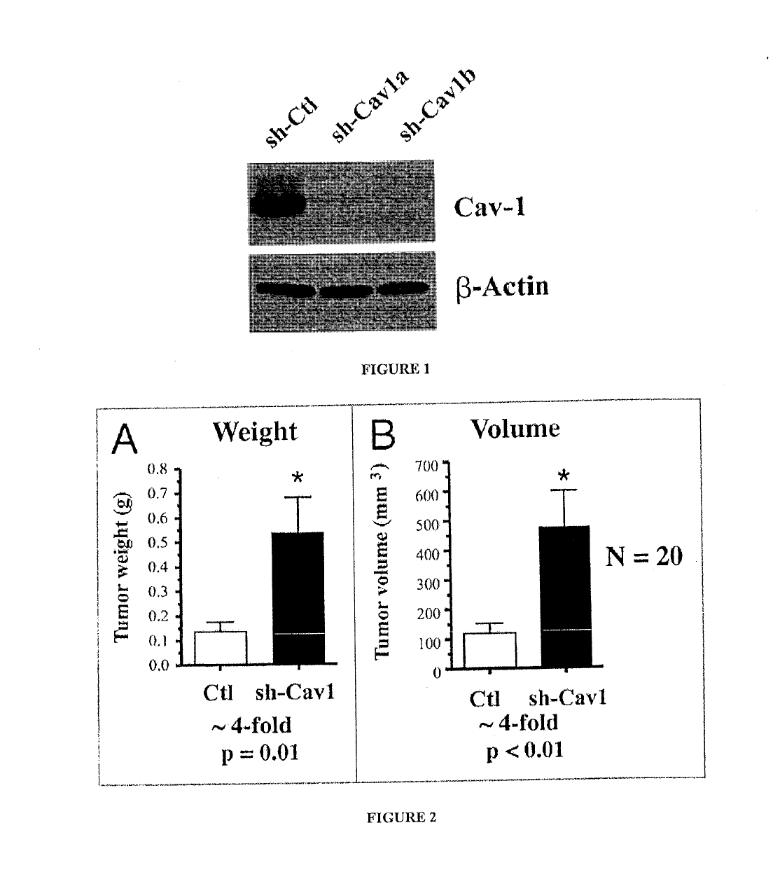

FIG. 1 illustrates targeted knock-down of Cav-1 protein expression in hTERT-Fibroblasts. To dissect the role of Cav-1 in promoting the growth of triple negative breast cancers, we have created a matched set of hTERT-immortalized human fibroblast cell lines (from parental hTERT-BJ1), either expressing an shRNA targeting Cav-1 or a control shRNA. This retroviral vector also contains GFP, so transduced cells were recovered by FACS sorting. Successful knock-down of Cav-1 was verified by Western blot analysis. The expression of beta-actin is shown as a control for equal protein loading, Ctl, control sh-RNA; sh-Cav1, harboring sh-RNA targeting Cav-1.

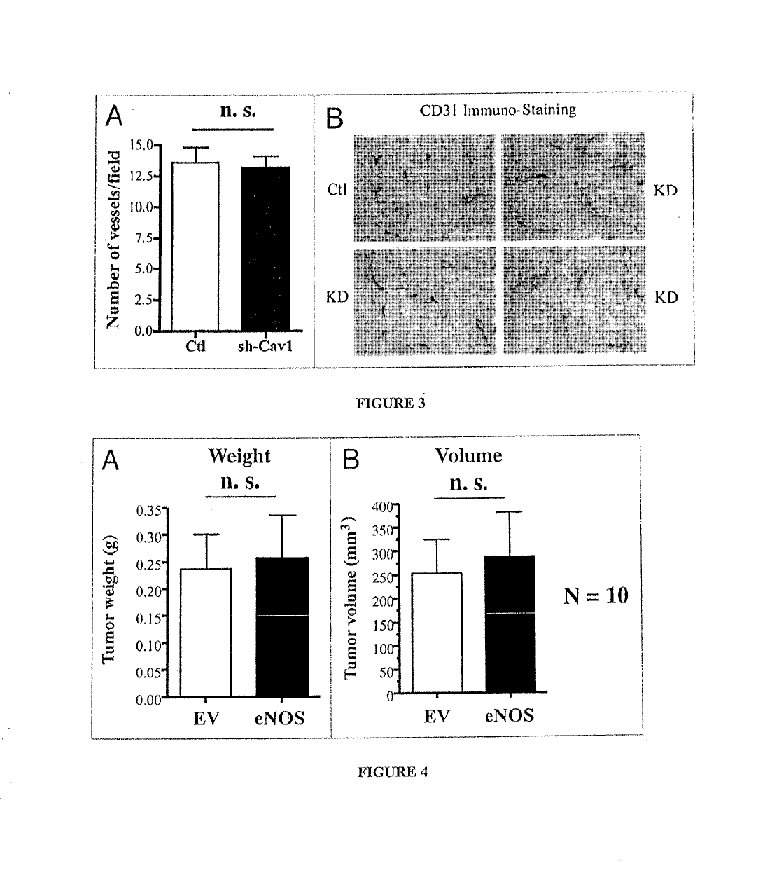

FIG. 3 illustrates targeted knock-down of Cav-1 in stromal fibroblasts does not affect tumor angiogenesis. Frozen sections from the tumors were cut and immuno-stained with anti-CD31 antibodies, and vessel density was quantitated (A). No significant increases in vessel density were observed, suggesting that the tumor promoting effects of the Cav-1 knock-down fibroblasts observed are independent of angiogenesis (n.s.=not significant; Representative images are shown in (B); Ctl=control sh-RNA; KD=harboring sh-RNA targeting Cav-1 (knock-down)).

FIG. 5 illustrates mitochondrial SOD2 significantly reverts the tumor promoting phenotype of Cav-1 deficient fibroblasts. Since loss of Cav-1 increases ROS production in stromal fibroblasts, the resulting oxidative stress, was modulated with SOD2 stably overexpressed in Cav-1 knock-down fibroblasts, using a lenti-viral vector with puromycin resistance. Cav-1 knock-down cells were transfected with the empty vector alone, in parallel. Then, these 2 fibroblast lines were co-injected with MDA-MB-231 cells into the flanks of nude mice. Overexpression of SOD2, a mitochondrial enzyme that deactivates super-oxide, was sufficient to reduce the tumor promoting effects of Cav-1 knock-down fibroblasts by nearly 2-fold. An asterisk indicates that p=0.01 (B). The overexpression of SOD2 was validated by Western blot analysis (A). The expression of beta-actin is shown as a control for equal protein loading (N≧9 flank injections for each experimental group).

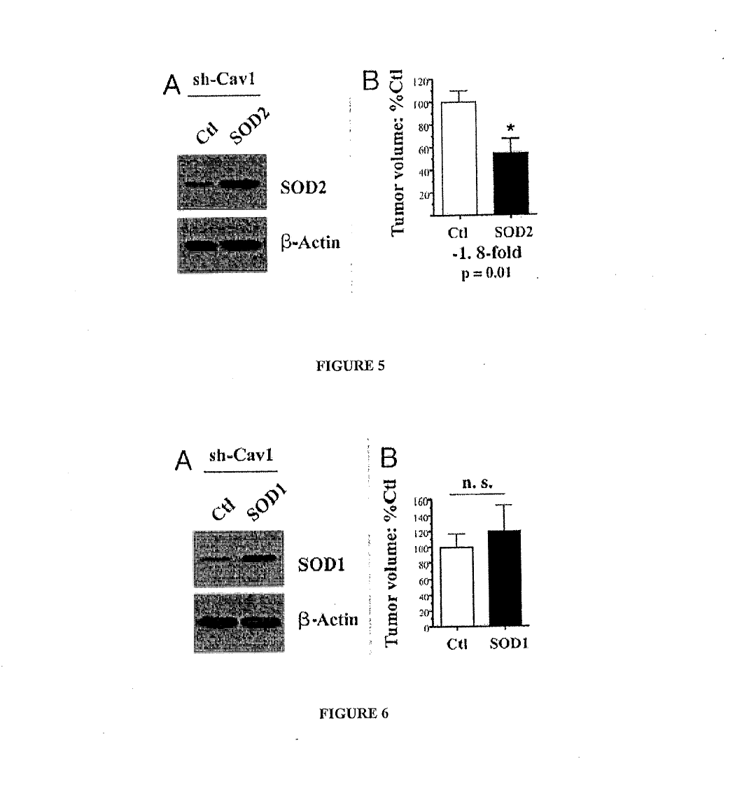

FIG. 5B shows that recombinant expression of mitochondrially-targeted SOD2 was sufficient to significantly revert the tumor promoting effects of a Cav-1 deficient tumor micro-environment, resulting in a near 2-fold reduction in tumor volume (FIG. 5B). In contrast, recombinant expression of cytoplasmic soluble SOD1 was not sufficient to revert the tumor promoting effects of a loss of Cav-1 (FIG. 6), further highlighting the specific role of mitochondrial oxidative stress in this process. Thus, these studies provide proof-of-principal that Cav-1 deficient hTERT fibroblasts can be used successfully as a genetically tractable system to identify the key factors that govern the tumor-promoting “lethal” effects of a Cav-1 deficient tumor micro-environment.

FIG. 6 illustrates that cytoplasmic soluble SOD1 does not revert the tumor promoting phenotype of Cav-1 deficient fibroblasts. To combat the oxidative stress, SOD1 was stably overexpressed in Cav-1 knock-down fibroblasts, using a lenti-viral vector with puromycin resistance, Cav-1 knock-down cells were transfected with the empty vector alone, in parallel. Then, these 2 fibroblast lines were co-injected with MDA-MB-231 cells into the flanks of nude mice. Overexpression of SOD1, a cytoplasmic soluble enzyme that deactivates super-oxide, was not sufficient to reduce the tumor promoting effects of Cav-1 knock-down fibroblasts. The overexpression of SOD1 was validated by Western blot analysis (A). The expression of beta-actin was shown as a control for equal protein loading (N≧8 flank injections for each experimental group; n.s.=not significant).

The proteomic profiles obtained from Cav-1 deficient and eNOS transfected fibroblasts were found to overlap with the transcriptional stromal profiles obtained from human breast caners. Transcriptional profiles of a large data set of human breast cancer patients whose tumors were subjected to laser-capture micro-dissection in classified based on stroma into three subgroups: Tumor stroma vs. normal stroma list; Recurrence stroma list; and Lymph-node (LN) metastasis stroma list. The tumor stroma vs. normal stroma list compared the transcriptional profiles of tumor stroma obtained from 53 patients to normal stroma obtained from 38 patients. Gene transcripts that were consistently upregulated in tumor stroma were selected and assigned a p-value, with a cut-off of p<0.05 (contains 6,777 genes). The recurrence stroma list compared the transcriptional profiles of tumor stroma obtained from 11 patients with tumor recurrence to the tumor stroma of 42 patients without tumor recurrence. Gene transcripts that were consistently upregulated in the tumor stroma of patients with recurrence were selected and assigned a p-value, with a cut-off of p<0.05 (contains 3,354 genes). And the lymph-node (LN) metastasis stroma list compared the transcriptional profiles of tumor stroma obtained from 25 patients with LN metastasis to the tumor stroma of 25 patients without LN metastasis. Gene transcripts that were consistently upregulated in the tumor stroma of patients with LN metastasis were selected and assigned a p-value, with a cut-off of p<0.05 (contains 1,182 genes). All three gene lists were then individually intersected with the proteomic profiles of Cav-1 deficient and eNOS-transfected fibroblasts. The results are shown in Table 4.

TABLE 4
Intersection of eNOS fibroblast proteomics with the
transcriptomeof human breast cancer tumor stroma.

| | | Recurrence- | Metastasis- |
|---|---|---|---|
| Gene symbol | Tumor stroma | Prone stroma | Prone stroma |
| ACO2 | 9.88E−18 | | |
| ALB | | | 3.95E−02 |
| ANXA2 | | | |
| APEX1 | | | 4.95E−02 |
| ATP5A1 | | | |
| BAG2 | | | |
| CAPZB | | | |
| COL6A2 | | | |
| DNAJA3 | 4.14E−04 | 1.30E−02 | |
| ENO1 | | | |
| GAPDH | | | |
| GDF2 | 5.20E−14 | 5.11E−03 | |
| GLUD1 | | | |
| HIST2H4B | | | |
| HNRNPA2B1 | | | |
| HSPA8 | | | |
| HSPA9 | | | |
| MATR3 | | | |
| PITRM1 | | | |
| PKM2 | | | 3.73E−02 |
| PRDX6 | | | 3.62E−02 |
| SCO2 | | | |
| SHMT2 | | 3.29E−02 | |
| SOD2 | 1.45E−11 | | |
| TRPC4AP | | | |
| VAT1 | 2.28E−11 | 1.76E−02 | |
| VIM | | | |
| WDR78 | 2.46E−08 | | |

Proteins that were transcriptionally upregulated in laser-capture microdissected human breast cancer tumor stroma are shown in BOLD. Those gene products that are associated with tumor recurrence or metastasis are shown in BOLD and are underlined. P-values are as shown.

Proteins that were transcriptionally upregulated in laser-capture microdissected human breast cancer tumor stroma are shown in BOLD. Those gene products that are associated with tumor recurrence or metastasis are shown in BOLD and are underlined. P-values are as shown.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A genetic model system for identifying genetic factors that govern the tumor promoting effects of cancer-associated cells, the genetic model system comprising human Cav-1 deficient fibroblast co-injected with a human cancer cell line into an immunodeficient animal and using proteomics to determine biomarkers that are expressed at altered levels relative to a control to thereby evaluate the presence or absence of oxidative stress.

2. (canceled)

3. (canceled)

4. (canceled)

5. A method for determining the prognosis of a cancer in a subject, the method comprising: (a) determining the expression level of at least one biomarker or a prognostic signature, said at least one biomarker or prognostic signature being associated with the prognosis of the cancer, wherein the at least one biomarker or the prognostic signature comprises one or more biological molecules which are associated with the prognosis of the cancer, in a sample of the cancer obtained from the subject; (b) comparing the level of the at least one biomarker or the prognostic signature expression in the cancer sample with the level of the at least one biomarker or the prognostic signature expression in a control sample, wherein said prognosis is made when the expression level of the at least one biomarker or a prognostic signature in the sample of cancer is greater than the expression level of the at least one biomarker or the prognostic signature in the control sample, wherein the one or more biological molecules are selected from the group consisting of AC02, ALB, ANPEP, ANXA2, APEX1, ATP5A1, BAG2, CALR, CALU, CAPZB, CDC42, COL1A1, COL6A1, COL6A2, CRABP2, CRTAP, DMGDH, DNAJA3, DNM1L, ENO1, ETFB, FBN1, FKBP9, GAPDH, GDF2, GLUD1, HIST2H4B, HNRNPA2B1, HSPA8, HSPA9, HSPB1, HSPD1, IDH2, KIAA1409, LDHA, LDHAL6B, LGALS1, LGALS3, LMNA, MATR3, MT1M, MYL6, NDUFA5, NDUFS3, P4HA1, P4HA2, PITRM1, PKM2, PLOD1, PRDX1, PRDX4, PRDX6, PSME1, RAPIA, RCN1, RPLP2, S100A13, SC02, SERPI H1, SHMT2, SOD2, SY J2BP, TPM1, TPM4, TRPC4AP, TX DC5, UQCRFS 1, VAT1, VIM, WDR78, XRCC6BP1, YWHAB, YWHAZ and combinations thereof.

6. (canceled)

7. A prognostic method for breast cancer, the method comprising: (a) providing a biological test sample from a subject afflicted with breast cancer or suspected of having breast cancer; (b) determining a level of at least one biomarker in the test sample that is associated with the prognosis of the breast cancer; (c) comparing the level of said at least one biomarker in said test sample to the level of the biomarker in a control sample, wherein an elevated level of the biomarker in said test sample relative to the level of the biomarker in said control sample is a prognostic indicator of the course of breast cancer disease in said subject.

8. A method of monitoring the progression of breast cancer in a subject, the method comprising: (a) obtaining a first sample from a subject at a first time point and a second sample from said subject at a second time point; (b) determining the level of at least one biomarker in said first and second samples; (c) comparing the level of said at least one biomarker in said first sample to the level of said biomarker in said second sample, wherein an elevated level of the at least one biomarker in said second sample relative to the level in said first sample is an indication that the cancer has progressed in said subject.

9. (canceled)

10. A method for treating a neoplastic disease in a patient, comprising (a) obtaining a sample of stromal cells adjacent to the neoplasm from the neoplastic disease patient; (b) determining the level of caveolin-1 (Cav-1) protein expression in the stromal cells of the sample and comparing the level of Cav-1 protein expression in the stromal cells of the sample with the level of Cav-1 protein expression in a control; (c) predicting if the neoplasm will respond effectively to treatment with an anti-angiogenic agent, wherein said prediction is made when the level of Cav-1 protein expression in the stromal cells of the sample is lower than the level of Cav-1 protein expression in the control; and administering to said patient a therapeutically effective amount of an anti-neoplastic agent.

11. (canceled)

12. (canceled)

13. (canceled)

14. (canceled)

15. (canceled)


SEQUENCE LISTING (2 sequences)

SEQ ID NO: 1 - DNA, 21 bp, Homo sapiens
gctgagcgagaagcaagtgta

SEQ ID NO: 2 - DNA, 21 bp, Homo sapiens
tgggcagttgtaccatgcatt